(19) United States
(12) Patent Application Publication
(10) Pub. No.: US 2010/0076252 A1
(43) Pub. Date: Mar. 25, 2010

(54) PYRO/PIEZO SENSOR AND STIMULATOR HYBRID CIRCUIT

(75) Inventors: Reinhold Henke, Plymouth, MN (US); Peter Stasz, Mounds View, MN (US)

Correspondence Address: SCHWEGMAN, LUNDBERG & WOESSNER, P.A., P.O. BOX 2938, MINNEAPOLIS, MN, 55402, US

(73) Assignee: Dymedix Corporation, Shoreview, MN (US)

(21) Appl. No.: 12/562,959

(22) Filed: Sep. 18, 2009

Related U.S. Application Data
(60) Provisional application No. 61/098,398, filed on Sep. 19, 2008.
(60) Provisional application No. 61/098,394, filed on Sep. 19, 2008.

(51) Int. Cl. A61M 21/02

(52) U.S. Cl. 600/28; 600/27; 600/26

(57) ABSTRACT

This document discusses, among other things, an apparatus and method for receiving respiration information of a patient and for providing a stimulation to the patient using a hybrid circuit. The hybrid circuit includes a control input for receiving a control signal having a first and second state. A sense switch of the hybrid circuit can provide respiration information from a pyro/piezoelectric film sleep sensor and stimulator to a closed loop neuromodulator in response to the first state. A stimulation switch of the hybrid circuit can provide stimulation energy from the closed loop neuromodulator to the pyro/piezoelectric film sensor and stimulator in response to the second state. The hybrid circuit can couple to the pyro/piezoelectric film sensor and stimulator using a single wire pair.

### PRIORITY AND RELATED APPLICATIONS

This application claims the benefit under 35 U.S.C. 119(e) of U.S. Provisional Patent Application Ser. No. 61/098,398, filed on Sep. 19, 2008 and U.S. Provisional Patent Application Ser. No. 61/098,394, filed on Sep. 19, 2008, the disclosures of which are incorporated by reference herein in their entirety.

### TECHNICAL FIELD

This invention relates generally to the field of neurological disorders and more specifically to the area of sleep medicine and yet even more specifically to the area of sleep therapy for patients who suffer from sleep disorders. More particularly, the present invention relates to a hybrid circuit for a pyro/piezo sensor and stimulator.

### BACKGROUND

Sleep disorders have recently become the focus of a growing number of physicians. Sleep disorders include obstructive sleep apnea, central sleep apnea, complex sleep apnea, snoring, restless leg syndrome (RLS), periodic limb movement (PLM), sudden infant death syndrome (SIDS), and related neurological and physiological events or conditions occurring during sleep. Many hospitals and clinics have established sleep laboratories (sleep labs) to diagnose sleep disorders. In the sleep laboratories, practitioners use instrumentation to monitor and record a patient's sleep states, stages and behaviors during sleep. Practitioners rely on these recordings to diagnose patients and prescribe proper therapies. As a result of the sleep diagnosis in the sleep laboratory, the sleep practitioner prescribes a sleep therapy device for the sleep patient to use during regular sleep at home or any other patient sleeping environment.

The primary goal of addressing sleeping disorders is to help a person sleep better. The secondary goal of addressing sleeping disorders is to help a person live longer. It is well known that various undesirable behaviors often occur during sleep such as snoring, apnea episodes, abnormal breathing, Bruxism (teeth clenching and grinding) and the like. It is further known that these disorders and other undesirable behaviors may not only lead to insufficient amounts of sleep or fatigue but are also linked to co-morbidities such as obesity, high blood pressure, diabetes, cardiac diseases, stroke all of which lead to a pre-mature death. Even SIDS is suspected to be linked to an infant's sleep disorder.

Serious efforts are being made to reduce or eliminate these undesirable disorders and behaviors in part because of these co-morbidity concerns.

It is well known that several states of sleep exist and involve varying levels of consciousness. It is further well known that the beneficial effects of sleep improve when it is uninterrupted. To the extent that the above controllers/systems and associated stimulation devices alter a patient's sleep state or in a worst-case scenario actually awaken a patient, the devices have gone too far. While they may have stopped the undesirable behavior, they have neither helped a person sleep better nor have they helped a person to live longer.

The goal of addressing sleeping disorders is often to help a person sleep better. It is well known that several states of sleep exist and involve varying levels of consciousness. It is further well known that the beneficial effects of sleep improve when it is uninterrupted.

### SUMMARY

Various examples of the present subject matter provide a hybrid circuit for a pyro/piezo sensor and stimulator that by means of a control signal, switches between a sense signal path from a pyro/piezo sensor and stimulator located on the sleep study patient to a closed loop neuromodulator or from the closed loop neuromodulator back to the pyro/piezo sensor and stimulator affixed to the patient. The hybrid circuit separates the signal paths for sensing and stimulation from a pyro/piezo sensor and stimulator. A closed loop neuromodulator provides a control signal for the hybrid circuit. Arrangement of switches, load impedances and signal buffer control the flow of sensor and stimulation signal paths.

In Example 1, a hybrid circuit for receiving respiration information of a patient and for providing a stimulation to the patient includes a control input configured to receive a control signal from a closed loop neuromodulator, the control signal having a first state and a second state, a sense switch configured to provide the respiration information from a pyro/piezoelectric film sensor and stimulator to the closed loop neuromodulator in response to the first state of the received control signal, a stimulation switch configured to provide the stimulation energy from the closed loop neuromodulator to the pyro/piezoelectric film sensor and stimulator in response to the second state of the received control signal, and wherein the hybrid circuit is configured to couple to the pyro/piezoelectric film sensor and stimulator using a single wire pair.

In Example 2, the hybrid circuit of Example 1 optionally includes a sensing load impedance, coupled to the sensing switch, configured to suppress false sensing signals generated when the control signal switches the sensing switch.

In Example 3, the sensing load impedance of any one or more of Examples 1-2 optionally includes a 1 mega ohm resister connected in parallel with a 10 microfarad capacitor.

In Example 4, the hybrid circuit of any one or more of Examples 1-3 optionally includes a stimulation load impedance, coupled to the stimulation switch, configured to suppress transmission of false stimulation signals to the pyro/piezoelectric film sensor and stimulator when the control signal switches the stimulation switch.

In Example 5, the stimulation load impedance of any one or more of Examples 1-4 optionally includes a 1 kilo ohm resistor connected in parallel with a 0.01 microfarad capacitor.

In Example 6, a system for treating a patient with a sleep disorder includes a pyro/piezoelectric film sensor and stimulator configured to detect respiration information of the patient and to provide stimulation to the patient, a closed loop neuromodulator configured to provide a control signal to a hybrid circuit, to receive the respiration information from the hybrid circuit, and to provide the stimulation energy to the hybrid circuit, the hybrid circuit including a control input configured to receive the control signal from a closed loop neuromodulator, the control signal having a first state and a second state, a sense switch configured to provide the respiration information from the pyro/piezoelectric film sensor and stimulator to the closed loop neuromodulator in response to the first state of the received control signal, and a stimulation switch configured to provide the stimulation energy from the closed loop neuromodulator to the pyro/piezoelectric film sensor and stimulator in response to the second state of the received control signal, wherein the hybrid circuit is configured to couple to the pyro/piezoelectric film sensor and stimulator using a single wire pair, and wherein the stimulation is provided to the patient using the stimulation energy.

In Example 7, the hybrid circuit of Example 6 is optionally configured to switch a coupling of the single wire pair between a sensing output of the hybrid circuit and a stimulation input of the hybrid circuit.

In Example 8, the hybrid circuit of any one or more of Examples 6-7 optionally includes a sensing switch coupled to the sensing output, the sensing switch configured to couple the single wire pair to the sensing output in response to the first state of the control signal.

In Example 9, the hybrid circuit of any one or more of Examples 6-8 optionally includes a sensing load impedance, coupled to the sensing switch, configured to suppress false sensing signals generated when the hybrid circuit switches the single wire pair.

In Example 10, the sensing load impedance of any one or more of Examples 6-9 optionally includes a 1 mega ohm resister connected in parallel with a 10 microfarad capacitor.

In Example 11, the hybrid circuit of any one or more of Examples 6-10 optionally includes a stimulation switch coupled to the simulation input, the stimulation switch configured to couple the single wire pair to the stimulation input in response to the second state of the control signal.

In Example 12, the hybrid circuit of any one or more of Examples 6-11 optionally includes a stimulation load impedance, coupled to the stimulation switch, configured to suppress transmission of false stimulation signals to the pyro/piezoelectric sleep film sensor and stimulator when the hybrid circuit switches the single wire pair.

In Example 13, the stimulation load impedance of any one or more of Examples 6-12 optionally includes a 1 kilo ohm resistor connected in parallel with a 0.01 microfarad capacitor.

In Example 14, the pyro/piezoelectric film sensor and stimulator of any one or more of Example 6-14 is optionally configured to provide tactile stimulation to the patient.

In Example 15, the pyro/piezoelectric film sensor and stimulator of any one or more of Example 6-15 is optionally configured to provide acoustical stimulation to the patient.

In Example 16, a method includes receiving a control signal at a hybrid circuit, the control signal having a first state and a second state, providing respiration information from a pyro/piezoelectric film sensor and stimulator to a closed loop neuromodulator in response to the first state of the control signal, providing stimulation energy from the closed loop neuromodulator to the pyro/piezoelectric film sensor and stimulator in response to the second state of the control signal, and wherein the providing the respiration information and the providing the stimulation energy includes using a single wire pair coupling the pyro/piezoelectric film sensor and stimulator to the hybrid circuit.

In Example 17, the providing the respiration information of Example 16 optionally includes switching a sense relay to couple the single wire pair to a respiration input of the closed loop neuromodulator in response to the first state of the control signal.

In Example 18, the switching the sense relay of any one or more of Example 16-17 optionally includes attenuating signal noise using a stimulation load impedance coupled to the sense relay.

In Example 19, the providing the stimulation energy of any one or more of Examples 16-18 optionally includes switching a stimulation relay to couple the single wire pair to a stimulation energy output of the closed loop neuromodulator in response to the second state of the control signal.

In Example 20, the switching the stimulation relay of any one or more of Examples 16-19 optionally includes attenuating signal noise using a sense load impedance coupled to the stimulation relay.

While the present disclosure is directed toward treatment of sleep disorders, further areas of applicability may become apparent from the description provided herein. It should be understood that the description and specific examples are intended for purposes of illustration only and are not intended to limit the scope of the present disclosure.

### BRIEF DESCRIPTION OF THE DRAWINGS

The forgoing features, objects and advantages of the invention will become apparent to those skilled in the art from the following detailed description, especially when considered in conjunction with the accompanying drawings in which like the numerals in the several views refer to the corresponding parts:

FIG. 1 is a block diagram representation of the hybrid circuit for a pyro/piezo sensor and stimulator in accordance with one embodiment of the present invention;

FIG. 2 is an electrical connection diagram for the hybrid circuit of FIG. 1;

FIG. 3 is an electrical schematic diagram of the hybrid circuit of FIG. 1;

FIG. 4 is an electrical timing diagram for the hybrid circuit of FIG. 1;

### DETAILED DESCRIPTION

The following detailed description relates to a hybrid circuit for a pyro/piezo sensor and stimulator for use in treating patients with sleep disorders. The hybrid circuit for a pyro/piezo sensor and stimulator is more particularly designed for stimulating a patient to interrupt and terminate an undesired sleep behavior or condition, such as snoring, sleep apnea, sudden infant death syndrome (SIDS) and others and with the same device also sense the respiratory activity of the same patient.

The assignee, Dymedix of Shoreview, Minnesota has also filed for U.S. Provisional Patent Application Ser. No. 61/098,394 filed Sep. 19, 2008 entitled Pyro/Piezo Sensor and Stimulator, the contents of which are hereby incorporated by reference herein in their entirety.

The inventors have recognized the need for, and have developed apparatus and methods for better sleep therapy devices including, but not limited to, a hybrid circuit for a pyro/piezo sensor and stimulator, which combines the separate sense and stimulation signals originating from a closed loop neuromodulator, a hybrid circuit for a pyro/piezo sensor and stimulator, which separates the sense and stimulation signals originating at a pyro/piezo sensor and stimulator, a hybrid circuit for a pyro/piezo sensor and stimulator, which carries both sense and stimulation, signals on the same wire pair, and an apparatus that has as least one less set of wire pairs being attached to the sleep patient.

A pyro/piezo sensor and stimulator is more particularly described in U.S. Provisional Patent Application Ser. No. 61/098,394 filed Sep. 19, 2008 entitled Pyro/Piezo Sensor and Stimulator, the contents of which are hereby incorporated by reference herein in their entirety, and which was concurrently filed with the present priority document. As described, a hybrid circuit for a pyro/piezo sensor and stimulator and the pyro/piezo sensor and stimulator may be used in conjunction with a suitable controller. The sensor transmits respiratory information to the controller that then analyzes the information and may trigger the piezoelectric stimulator depending on the information received.

Described herein is a hybrid circuit for a pyro/piezo sensor and stimulator including plurality of switches and buffers arranged in a way as to provide, on one side, a single wire pair that carries sensor and stimulation signals from and to a pyro/piezo sensor and stimulator affixed to the patient and on the other side, a single wire pair that carries sensor signals to a closed loop neuromodulator input and another single wire pair that carries stimulation signals from the closed loop neuromodulator output. A control signal received from a closed loop neuromodulator determines the direction of the signal transfer, either from the pyro/piezo sensor and stimulator to a closed loop neuromodulator or from the closed loop neuromodulator to the pyro/piezo sensor and stimulator.

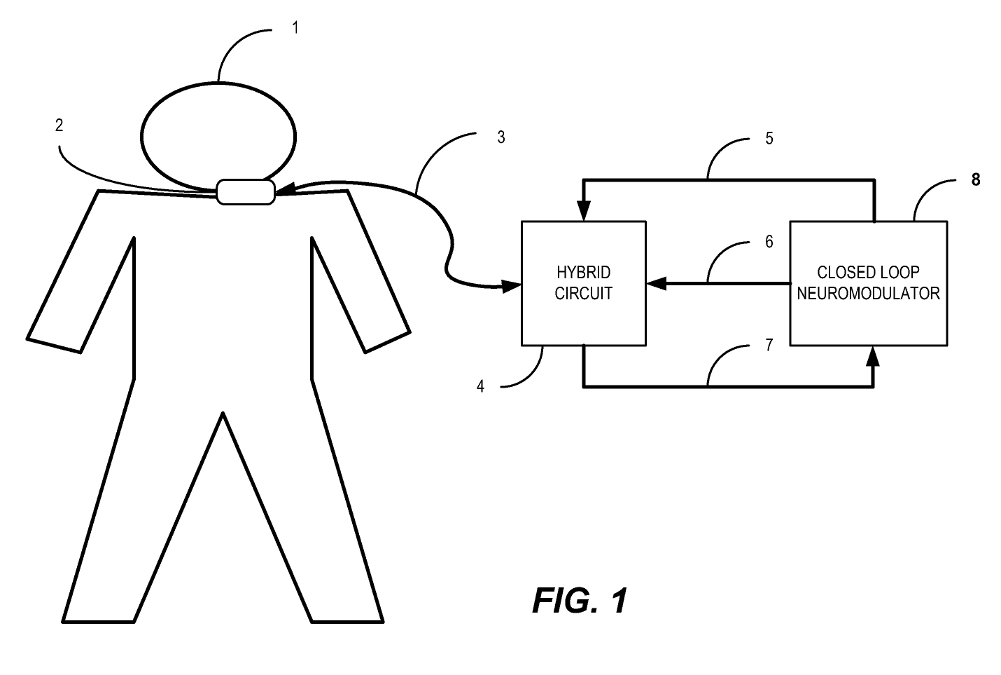

The following detailed description also includes discussion of sleep therapy devices, such as a closed loop neuromodulator, or a snore therapy device. A closed loop neuromodulator is described in U.S. patent application Ser. No. 12/583,581 filed Aug. 21, 2009, the entire disclosure of which is incorporated herein by reference. Additionally, elements of a hybrid circuit for a pyro/piezo sensor and stimulator are discussed including switches, wire terminations, load impedances, buffers and ground references. The present invention can be readily understood from FIGS. 1 through 4.

Referring to FIG. 1, there is indicated generally by numeral 1 a typical sleep therapy patient who has been outfitted with a pyro/piezo sensor and stimulator 2 to measure the patient's respiratory activity and to stimulate the patient in response to a dosed therapy signal from a closed loop neuromodulator 8. A pair of wire leads 3 connects the pyro/piezo sensor and stimulator 2 to the sense and stimulation terminals of a hybrid circuit 4. The hybrid circuit connects, via its sense only terminal connection 7, to a closed loop neuromodulator 8. The hybrid circuit 4 connects, via its stimulation only terminal connection 5, to the closed loop neuromodulator 8. The closed loop neuromodulator 8 connects, via the sense/stimulation control terminal 6, to the hybrid circuit 4 to select either sensing or stimulating operation of the sensor/stimulator device.

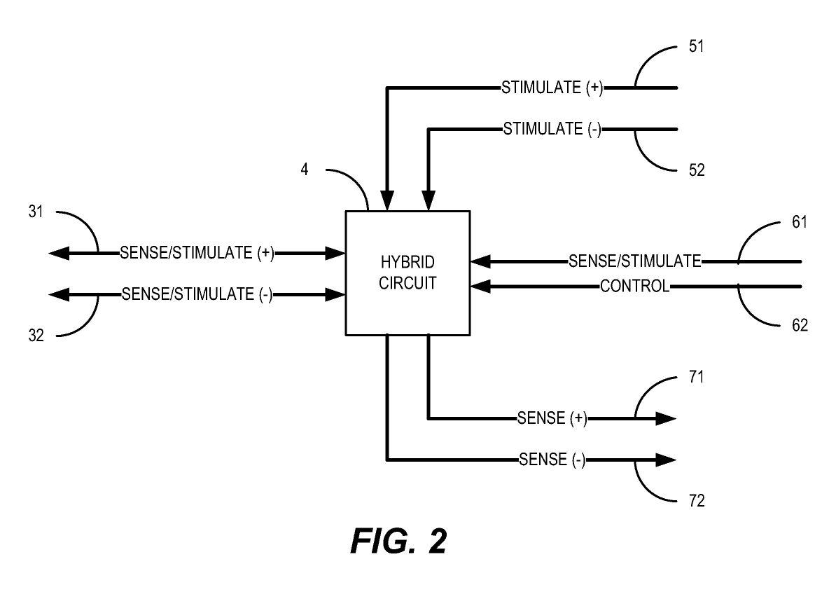

Referring to FIG. 2, there is shown an electrical connection diagram of the hybrid circuit 4. It receives and transmits its sensor output and stimulation input signal from and to the pyro/piezo sensor and stimulator via wire connections labeled “sense/stimulate (+)” 31 and “sense/stimulate (−)” 32. The closed loop neuromodulator selects between receiving a sense signal from the pyro/piezo sensor and stimulator 2 and sending a stimulation signal to the pyro/piezo sensor and stimulator 2 via the “sense/stimulation control” wire 61 and the “sense/stimulation reference” wire 62. When the hybrid circuit 4 is selected by the closed loop neuromodulator 8 to be in a sense mode by a control signal or the sense/stimulation control wire 61, then the hybrid circuit 4 passes the sense signal, via the sense (+) and sense (−) wire connections, to the closed loop neuromodulator 8. When the hybrid circuit 4 is selected by the closed loop neuromodulator 8 to be in stimulate mode, via a control signal on wire 61, then the closed loop neuromodulator 8 passes the stimulation signal via the “stimulate (+)” and “stimulate (−)” wire connections 51 and 52, respectively, to the hybrid circuit 4. The hybrid circuit then passes the stimulation signal from the closed loop neuromodulator 8, via the “sense/stimulation (+)” and “sense/stimulation (−)” wires 31 and 32 to the pyro/piezo sensor and stimulator 2.

To maintain a sufficient amount of common mode rejection needed to ensure the proper operation of the sensitive input stage of the closed loop neuromodulator 8, the sense and stimulation wire connections from the sensor/stimulator device 2 to the hybrid circuit 4, and between the hybrid circuit 4 and the closed loop neuromodulator 8 may be physically balanced and electrically symmetrical.

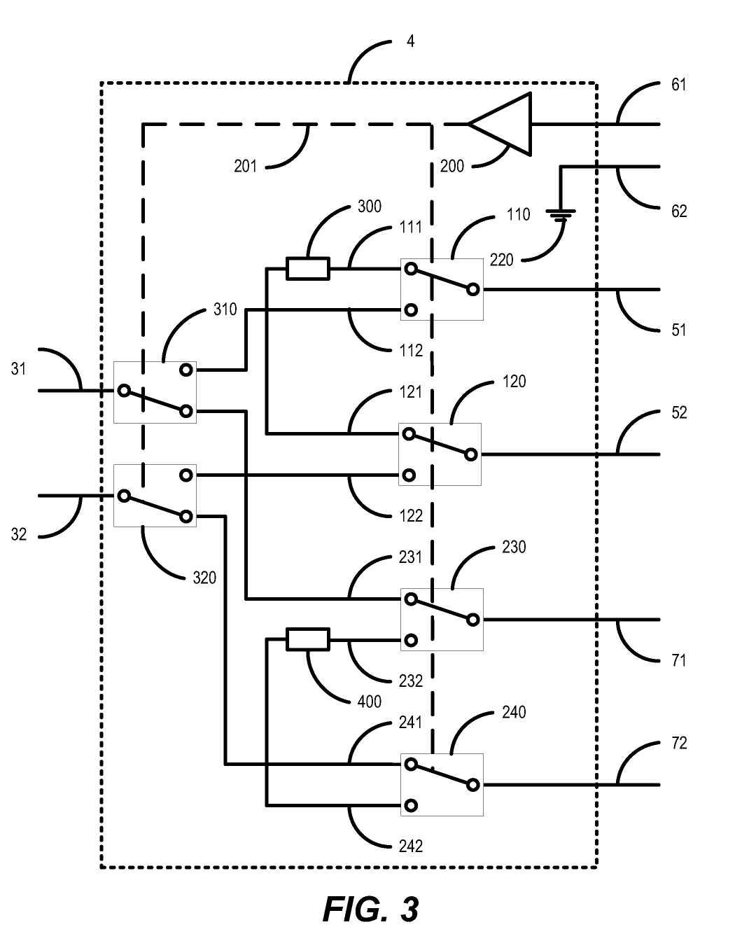

Referring to FIG. 3, there is shown an electrical schematic diagram of the hybrid circuit 4. It is seen to comprise a set of switches 110, 120, 230, 240, 310, and 320, a logic buffer 200, a plurality of internal wiring connections 111, 112, 121, 122, 231, 232, 141 and 242, a ground reference 220, a plurality of load impedances 300 and 400, and a multitude of wire terminations 31, 32, 51, 52, 61, 62, 71 and 72.

In this specific embodiment, switches are shown as single-pole, dual-throw (SPDT) types. This switch configuration has been found to work optimally but other configurations may be used just as well.

It will be clear to persons skilled in the art that the same functionality of the hybrid circuit for a pyro/piezo sensor and stimulator may be achieved with otherwise configured switches as well. For example: Single Pole Single Throw (SPST), Dual Pole Single Throw (DPST), Single Pole Dual Throw (SPDT) and Dual Pole Dual Throw (DPDT). Also, the switches may be implemented in form of different component technologies for example: Mechanical relays, Solid State Relays (SSR's), Opto Couplers, Bipolar Junction Transistors (BJT's), Metal Oxide Semiconductor Field Effect Transistors (MOS-FET's) and many other similar technologies and types.

In this specific embodiment, the switches 110, 120, 230, 240, 310, and 320 are preferably of a solid-state relay type, such as the PS7122A-1C available from California Eastern Laboratories of Santa Clara, Calif. Other solid state relays may be used as well. For example: The form-C may easily be replaced by a form-A or a form-B solid relay switch configuration.

In this specific embodiment, the buffer 200 may be one of six of the DM7405 Hex Inverter with open collector outputs available from National Semiconductor of Santa Clara, Calif. Its output is represented by dashed lines 201 showing that it controls the switch state of devices, 120, 220, 230, 240, 310 and 320.

The load impedances 300 and 400 may comprise discrete resistors and capacitors.

In one example, the load impedance 300 is constructed of a 10 Mohm resistor in parallel with a 0.01 uF capacitor. These values have been found to work optimally but other values may be used as well. A load impedance 400 may be constructed using a 1 kohm resistor in parallel with a 0.01 uF capacitor.

The load impedance 300 reduces the sending of spurious signals into the sensitive input stage of the closed loop neuromodulator that could be construed as actual sense signals that might require further analysis. With this load impedance in place, the transition from sensing mode to stimulation mode and vice versa will not create a significant spurious signal the closed loop neuromodulator input stage.

The Load impedance 400 also reduces the sending of spurious signals into the pyro/piezo sensor and stimulator that could be construed as actual stimulation signals that may cause the patient to prematurely respond to an erroneous stimulus signal. With load impedance 400 in place, transition from sensing to stimulation and vice versa will not create a significant noise signal applied to the pyro/piezo sensor and stimulator 2.

Following is a detailed description of the electrical schematic diagram of the hybrid circuit for a pyro/piezo sensor and stimulator 4.

The sense/stimulation control wire 61 controls the direction of the flow of sense and stimulation signals through the hybrid circuit.

As drawn, the hybrid circuit in FIG. 3 is in the “sense” state.

In the sensing state the sense signal flows from the attached pyro/piezo sensor and stimulator attached to the patient, via the balanced input sense/stimulate (+) 31 and sense/stimulate (−) 32 balanced wire pairs into the hybrid circuit. They connect to the common terminal of the SPDT relay switches 310 and 320, respectively.

The normally closed terminals of the relay switches 310 and 320 connect, via a set of balanced wire pairs 231 and 241, respectively, to the normally closed switch terminals of another set of SPDT relay switches 230 and 240, respectively.

The common terminals of the relay switches 230 and 240 respectively connect to the attached closed loop neuromodulator 8 via the sense (+) 71 and the sense (−) 72 balanced wire pairs respectively.

Also, in the sense state, the sense load impedance 300 is connected via the normally closed terminal of the relay switches 110 and 120, respectively, via the wiring connections 111 and 121 respectively.

The common terminals of the relay switches 110 and 120 respectively connect to the attached closed loop neuromodulator 8 via the stimulation (+) 51 and the stimulation (−) 52 balanced wire pairs.

In the stimulation state the stimulation signal flows from the attached closed loop neuromodulator via the balanced input stimulate (+) 51 and stimulate (−) 52 balanced wire pairs into the hybrid circuit of FIG. 3.

The balanced input wire pairs 51 and 52 labeled “stimulate (+)” connect to the common terminal of the SPDT relay switches 110 and 120 respectively.

The open terminals of the relay switches 110 and 120 connect, via a set of balanced wire pairs 112 and 122, respectively, to the normally open switch terminals of the SPDT relay switches 310 and 230 respectively. The common terminals of the relay switches 310 and 320 respectively connect to the attached pyro/piezo sensor and stimulator 2 via the sense/stimulate (+) 71 and the sense/stimulate (−) 32 balanced wire pairs respectively.

Also, in the stimulation state, the stimulate load impedance 400 is connected via the normally closed terminals of the relay switches 230 and 240 respectively via the wiring connections 232 and 242, respectively.

The common terminals of the relay switches 230 and 240 respectively connect to the attached closed loop neuromodulator via the “sense (+)” and the “sense (−)” balanced wire pairs 71 and 72.

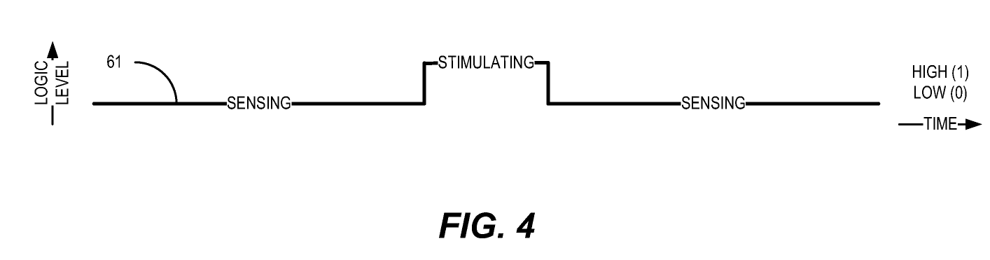

Referring to FIG. 4, there is shown a timing diagram of the hybrid circuit of FIG. 3, and more specifically the timing for the selection between receiving a sensing signal only and transmitting a stimulation signal only. There is indicated by numeral 61 a sense/stimulate control signal. The sense/stimulate signal 61 is represented by a logic level signal. The logic levels may take on either a logic high state (“1”) or a logic low state (“0”). The high state indicates “stimulating” which means that a stimulation signal is sent from the closed loop neuromodulator 8, via the hybrid circuit 4, to the sensing stimulator 2 and that the patient is currently being subjected to a precisely dosed stimulation signal originating at the closed loop neuromodulator 8. The low state indicates “sensing”, which means that a sensed signal is sent from the pyro/piezo sensor and stimulator 2 via the hybrid 4 to the closed loop neuromodulator 8 for further analysis.

In application, in one instant, a closed loop neuromodulator may transmit precisely dosed sleep therapy signal in form of an alternating current (AC) via wire pair 5 (FIG. 1) to the pyro/piezo sensor and stimulator for patient stimulation. In another instant, a closed loop neuromodulator may receive a sensor signal in form of an alternating current (AC) via wire pair 7. A logic control signal wire pair 6 provides the selection between sensing biological information and stimulating the patient with therapy signals.

The present invention functions to separate the sense and stimulation signals originating at a pyro/piezo sensor and stimulator and carries sense and stimulation, signals on the same wire pair. Thus, one less set of wire pairs need be attached to the sleep patient.

This invention has been described herein in considerable detail in order to comply with the patent statutes and to provide those skilled in the art with the information needed to apply the novel principles and to construct and use such specialized components as are required. However, it is to be understood that the invention may be carried out by specifically different equipment and devices, and that various modifications, both as to the equipment and operating procedures, may be accomplished without departing from the scope of the invention itself.

The description of the various embodiments is merely exemplary in nature and, thus, variations that do not depart from the gist of the examples and detailed description herein are intended to be within the scope of the present disclosure. Such variations are not to be regarded as a departure from the spirit and scope of the present disclosure.

The description of the various embodiments is merely exemplary in nature and, thus, variations that do not depart from the gist of the examples and detailed description herein are intended to be within the scope of the present disclosure. Such variations are not to be regarded as a departure from the spirit and scope of the present disclosure.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventor also contemplates examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A hybrid circuit for receiving respiration information of a patient and for providing a stimulation to the patient, the hybrid circuit comprising:
a control input configured to receive a control signal from a closed loop neuromodulator, the control signal having a first state and a second state;
a sense switch configured to provide the respiration information from a pyro/piezoelectric film sensor and stimulator to the closed loop neuromodulator in response to the first state of the received control signal;
a stimulation switch configured to provide the stimulation energy from the closed loop neuromodulator to the pyro/piezoelectric film sensor and stimulator in response to the second state of the received control signal; and
wherein the hybrid circuit is configured to couple to the pyro/piezoelectric film sensor and stimulator using a single wire pair.

2. The hybrid circuit of claim 1, including a sensing load impedance, coupled to the sensing switch, configured to suppress false sensing signals generated when the control signal switches the sensing switch.

3. The hybrid circuit of claim 2, wherein the sensing load impedance includes a 1 mega ohm resister connected in parallel with a 10 microfarad capacitor.

4. The hybrid circuit of claim 1, including a stimulation load impedance, coupled to the stimulation switch, configured to suppress transmission of false stimulation signals to the pyro/piezoelectric film sensor and stimulator when the control signal switches the stimulation switch.

5. The hybrid circuit of claim 4, wherein the stimulation load impedance includes a 1 kilo ohm resistor connected in parallel with a 0.01 microfarad capacitor.

6. A system for treating a patient with a sleep disorder, the system comprising:
a pyro/piezoelectric film sensor and stimulator configured to detect respiration information of the patient and to provide stimulation to the patient;
a closed loop neuromodulator configured to provide a control signal to a hybrid circuit, to receive the respiration information from the hybrid circuit, and to provide the stimulation energy to the hybrid circuit, the hybrid circuit including:
a control input configured to receive the control signal from a closed loop neuromodulator, the control signal having a first state and a second state;
a sense switch configured to provide the respiration information from the pyro/piezoelectric film sensor and stimulator to the closed loop neuromodulator in response to the first state of the received control signal; and
a stimulation switch configured to provide the stimulation energy from the closed loop neuromodulator to the pyro/piezoelectric film sensor and stimulator in response to the second state of the received control signal;
wherein the hybrid circuit is configured to couple to the pyro/piezoelectric film sensor and stimulator using a single wire pair; and
wherein the stimulation is provided to the patient using the stimulation energy.

7. The system of claim 6, wherein the hybrid circuit is configured to switch a coupling of the single wire pair between a sensing output of the hybrid circuit and a stimulation input of the hybrid circuit.

8. The system of claim 7, wherein the hybrid circuit includes a sensing switch coupled to the sensing output, the sensing switch configured to couple the single wire pair to the sensing output in response to the first state of the control signal.

9. The system of claim 8, wherein the hybrid circuit includes a sensing load impedance, coupled to the sensing switch, configured to suppress false sensing signals generated when the hybrid circuit switches the single wire pair.

10. The system of claim 8, wherein the sensing load impedance includes a 1 mega ohm resister connected in parallel with a 10 microfarad capacitor.

11. The system of claim 7, wherein the hybrid circuit includes a stimulation switch coupled to the simulation input, the stimulation switch configured to couple the single wire pair to the stimulation input in response to the second state of the control signal.

12. The system of claim 11, wherein the hybrid circuit includes a stimulation load impedance, coupled to the stimulation switch, configured to suppress transmission of false stimulation signals to the pyro/piezoelectric sleep film sensor and stimulator when the hybrid circuit switches the single wire pair.

13. The system of claim 12, wherein the stimulation load impedance includes a 1 kilo ohm resistor connected in parallel with a 0.01 microfarad capacitor.

14. The system of claim 6, wherein the pyro/piezoelectric film sensor and stimulator is configured to provide tactile stimulation to the patient.

15. The system of claim 6, wherein the pyro/piezoelectric film sensor and stimulator is configured to provide acoustical stimulation to the patient.

16. A method comprising:
receiving a control signal at a hybrid circuit, the control signal having a first state and a second state;
providing respiration information from a pyro/piezoelectric film sensor and stimulator to a closed loop neuromodulator in response to the first state of the control signal;
providing stimulation energy from the closed loop neuromodulator to the pyro/piezoelectric film sensor and stimulator in response to the second state of the control signal; and
wherein the providing the respiration information and the providing the stimulation energy includes using a single wire pair coupling the pyro/piezoelectric film sensor and stimulator to the hybrid circuit.

17. The method of claim 16, wherein the providing the respiration information includes switching a sense relay to couple the single wire pair to a respiration input of the closed loop neuromodulator in response to the first state of the control signal.

18. The method of claim 17, wherein the switching the sense relay includes attenuating signal noise using a stimulation load impedance coupled to the sense relay.

19. The method of claim 16, wherein the providing the stimulation energy includes switching a stimulation relay to couple the single wire pair to a stimulation energy output of the closed loop neuromodulator in response to the second state of the control signal.

20. The method of claim 19, wherein the switching the stimulation relay includes attenuating signal noise using a sense load impedance coupled to the stimulation relay.
